(19) United States
(12) Patent Application Publication
(10) Pub. No.: US 2005/0149172 A1
(43) Pub. Date: Jul. 7, 2005

(54) Minimal injury resorbable stent

(76) Inventor: Ashish Varma, Galway (IE)

Correspondence Address: MEDTRONIC VASCULAR, INC.;IP LEGAL DEPARTMENT, 3576 UNOCAL PLACE, SANTA ROSA, CA, 95403, US

(21) Appl. No.: 10/740,805

(22) Filed: Dec. 22, 2003

(51) Int. Cl. A61F 2/06

(52) U.S. Cl. 623/1.38

(57) ABSTRACT

A minimal injury resorbable stent comprising an oriented resorbable material drawn to a ratio about 7-350 is disclosed. The drawn materials of the present invention have tensile strength of about 50-500 MPa and Young's modulus of about 2-300 GPa. The bioresorbable stents can have cylindrical shape and optionally further comprise one or more of a solvent, plasticizer, biologically active agent and modifier. Also disclosed is a method for manufacturing a minimal injury resorbable stent. The process comprises contacting a resorbable polymer with a solvent to form a polymer mixture, extruding said polymer mixture to form an extrudate, drawing said extrudate to a draw ratio in the range of about 7-350 to form a drawn extrudate and forming said stent from said drawn extrudate. The process optionally further comprises coagulating the extrudate and annealing the extrudate and/or stent.

### BACKGROUND OF THE INVENTION

1. Field of the Invention

The present invention relates to the field of resorbable stents.

Specifically, the present invention relates to minimal injury resorbable stents and a process for their manufacture.

2. Related Art

Stents have gained acceptance in the medical community as a device capable of supporting body lumens, such as blood vessels, that have become weakened or are susceptible to closure. Typically, a stent is inserted into a vessel of a patient after an angioplasty procedure has been performed to partially open up the blocked/stenosed vessel thus allowing access for stent delivery and deployment. After the catheter used to perform angioplasty has been removed from the patient, a tubular stent, maintained in a small diameter delivery configuration at the distal end of a delivery catheter, is navigated through the vessels to the site of the stenosed area. Once positioned at the site of the stenosis, the stent is released from the delivery catheter and expanded radially to contact the inside surface of the vessel. The expanded stent provides a scaffold-like support structure to maintain the patency of the region of the vessel engaged by the stent, thereby promoting blood flow. Physicians may also elect to deploy a stent directly at the lesion rather than carrying out a pre-dilatation procedure. This approach requires stents that are highly deliverable i.e. have low profile and high flexibility.

Various types of endovascular stents have been proposed and used as a means for preventing restenosis. A typical stent is a tubular device capable of maintaining the lumen of the artery open. One example includes metallic stents that have been designed and permanently implanted in arterial vessels.

Metallic stents have a low profile combined with high strength. Restenosis has been found to occur, however, in some cases despite the presence of the metallic stent. In addition, some implanted stents have been found to cause undesired local thrombosis. To address this, some patients receive anticoagulant and antiplatelet drugs to prevent local thrombosis or restenosis, however this prolongs the angioplasty treatment and increases its cost.

A number of non-metallic stents have been designed to address the concerns related to the use of permanently implanted metallic stents. U.S. Pat. No. 5,984,963 to Ryan, et al., discloses a polymeric stent made from resorbable polymers that degrades over time in the patient. U.S. Pat. No. 5,545,208 to Wolff, et al., discloses a polymeric prosthesis for insertion into a lumen to limit restenosis. The prosthesis carries restenosis-limiting drugs that are released as the prosthesis is resorbed. The use of resorbable polymers, however, has drawbacks that have limited the effectiveness of polymeric stents in solving the post-surgical problems associated with balloon angioplasty.

Polymeric stents are typically made from bioresorbable polymers.

Materials and processes typically used to produce resorbable stents result in stents with low tensile strengths and low modulus, compared to metallic stents of similar dimensions. The limitations in mechanical strength of the resorbable stents can result in stent recoil after the stent has been inserted. This can lead to a reduction in luminal area and hence blood flow. In severe cases the vessel may completely re-occlude. In order to prevent the recoil, polymeric stents have been designed with thicker struts (which lead to higher profiles) or as composites to improve mechanical properties. The use of relatively thick struts makes polymeric stents stiffer and decreases their tendency to recoil, but a significant portion of the lumen of the artery can be occupied by the stent. This makes stent delivery more difficult and can cause a reduction in the area of flow through the lumen. A larger strut area also increases the level of injury to the vessel wall and this may lead to higher rates of restenosis i.e. re-occlusion of the vessel.

Considerable research has been undertaken to develop resorbable stents that are satisfactory alternatives to metallic stents and are usable as an adjunct to angioplasty. However, there remains a need for materials and processes to produce resorbable stents with high tensile strengths, high modulus and low profile.

### SUMMARY OF THE INVENTION

It has been found that minimal injury resorbable stents having enhanced properties can be produced by introducing high levels of molecular alignment or orientation in the resorbable materials used in stent production. The present invention, therefore, relates to a method of controlling the morphology of the oriented resorbable materials and a method of manufacturing a low profile stent comprising the oriented resorbable materials.

An embodiment of the present invention relates to a minimal injury resorbable stent comprising an oriented resorbable material drawn to a ratio about 7-350. Alternatively, the materials are drawn to a ratio about 10-300. The drawn materials of the present invention have tensile strength of about 50-500 MPa and Young's modulus of about 2-300 GPa. The bioresorbable stents can have cylindrical shape and optionally further comprise one or more of a solvent, plasticizer, biologically active agent and modifier.

Another embodiment of the present invention relates to a method for manufacturing a minimal injury resorbable stent. The process comprises contacting a resorbable polymer with a solvent to form a polymer mixture, extruding said polymer mixture to form an extrudate, drawing the extrudate to a draw ratio in the range of about 7-350 to form a drawn extrudate and forming said stent from said drawn extrudate. The process produces drawn materials having tensile strength of about 50-500 MPa and Young's modulus of about 2-300 GPa. The process optionally further comprises coagulating the extrudate and annealing the extrudate and/or stent.

It is to be understood that both the foregoing general description and the following detailed description are exemplary and explanatory and are intended to provide further explanation of the invention as claimed.

### BRIEF DESCRIPTION OF THE FIGURES

The accompanying drawings, which are included to illustrate exemplary embodiments of the invention and are incorporated in and constitute a part of this specification, illustrate embodiments of the invention and together with the description serve to explain the principles of the invention. In the drawings:

FIG. 1 is a flowchart 100 showing the steps in the method of the present invention.

FIGS. 2-4 are flowcharts 200-400 showing optional additional steps in the method of the present invention.

The present invention will now be described with reference to the accompanying drawings. In the drawings, like reference numbers indicate identical or functionally similar elements. Additionally, the left-most digit(s) of a reference number identifies the drawing in which the reference number first appears.

### DETAILED DESCRIPTION OF THE INVENTION

Reference will now be made in detail to the embodiments of the present invention, examples of which are illustrated in the accompanying drawings.

Resorbable polymers extruded via conventional melt extrusion exhibit relatively high entanglement densities and this serves to limit the ultimate draw ratios (typically less than 10) and therefore the tensile strengths that may be achieved for these polymers. The traditional method to achieve chain orientation is based on drawing a melt extruded or molded polymer, often semi-crystalline, in the solid state at temperatures above its glass transition temperature. In this state, chain mobility is likely to be limited by entanglements as well as chain interactions and crystallinity. Therefore, it is also likely that entanglements, chain folds, and fragments of crystals survive the drawing process (which allows only limited drawing) and persist in the drawn polymer as defects which serve to limit the strength. The limitations imposed by entanglements and chain interactions would also apply to amorphous polymers.

The present invention involves the use of molecular alignment for enhancing the tensile strength and modulus of resorbable polymers suitable for making resorbable stents. The entanglement density of a high molecular weight resorbable polymer can be substantially reduced by means of a suitable solvent or diluent. The reduced entanglements allow for superdrawing and increased draw ratios. Specifically, the present invention relates to the use of gel (or solution) extrusion and drawing to significantly enhance the ultimate draw ratios, and hence the tensile strength, of resorbable polymers. Draw ratios of about 7-350 can be achieved via this technique. An increase in draw ratio decreases both the fraction of unoriented polymer and the ‘imperfection’ of molecular alignment. Higher draw ratios help to produce ‘ultra-oriented’ polymers with strengths that approach the theoretical strengths of covalent bonds. Higher tensile strengths serve to reduce the stent strut area and thus help to minimize vessel injury and vessel recoil and therefore help to improve procedure outcome.

In one embodiment, the present invention relates to a minimal injury resorbable stent comprising an oriented resorbable material drawn to high draw ratios. The materials of the present invention are drawn to ratios of about 7-350, or alternatively, 10-300. Resorbable is used herein to mean a material that dissolves over time. The process of dissolving can be by degradation, dissolution or by some other means by which the stent material dissolves into the body. Resorbable stents of the present invention are bioresorbable, or alternatively, biodegradable. Resorbable stents of the present invention comprise materials having a tensile strength of about 50-500 MPa, 75-400 MPa or 100-300 MPa. Alternatively, the stents comprise materials having tensile strengths of about 50-500 MPa and Young's modulus of about 2-300 GPa.

Tensile strength is the measure of the ability of a polymer to withstand pulling or expanding stresses. Tensile strength can be measured by any method known to one of ordinary skill in the art. One example is the testing method ASTM-D638-72 (available from ASTM International, West Conshohocken, Pa., 19428). As used herein, the term modulus, also known as the Young's modulus, is the stress per unit strain. The modulus is a measure of the stiffness of a material. Any method known to one of ordinary skill in the art can be used to measure modulus. For example, modulus can be measured using a tensile tester in accordance with methods well known in the art. Alternatively, a dynamic mechanical analyzer (DMA) is used to measure shear modulus, which can be converted to Young's modulus, as is well known to one skilled in the relevant art.

The resorbable stents of the present invention have low profile. The low profile allows the practitioner to use the stent in a variety of body lumens. For example, stents of the present invention can be used in blood-carrying vessels such as arteries and veins. More specifically, vessels in which the stents can be used include cardiovascular, neurovascular and peripheral blood carrying vessels.

Resorbable stents of the present invention comprise an oriented resorbable material. The term oriented is well known to one of ordinary skill in the art and is used herein to mean molecular alignment has been introduced into the material. Molecular orientation or alignment can be introduced in crystalline and amorphous phases of the material. Molecular orientation or alignment enhances the mechanical properties of the material. For example, introducing molecular alignment in a material increases the material's Young's modulus and tensile strength. One aspect of the present invention, therefore, is related to a method of inducing molecular alignment in a resorbable material to produce an oriented material, wherein the material has a greater Young's modulus and tensile strength than the unoriented material. The materials of the present invention can have any level of orientation or molecular alignment, so long as the material has higher modulus and tensile strength compared to the unoriented material. The enhanced mechanical properties of the oriented resorbable materials allow for the production of stents having high recoil resistance and low profile. Any method known to one skilled in the relevant art can be used to measure molecular alignment. For example, X-Ray analysis, can be used to determine the degree or amount of molecular alignment in the material. Alternatively, Fourier Transform Infrared (FTIR) spectroscopy is used, as is well known to one skilled in the relevant art.

Materials for use in the present invention include any resorbable material. In one example, the material comprises a resorbable polymer. Resorbable polymers for use in the present invention include but are not limited to polyesters, polyanhydrides, polyamides, polyurethanes, polyureas, polyethers, polysaccharides, polyamines, polyphosphates, polyphosphonates, polysulfonates, polysulfonamides, polyphosphazenes, a hydrogel, polylactides or polyglycolides. Specific examples of resorbable polymers include but are not limited to fibrin, collagen, polycaprolactone, poly(glycolic acid), poly(3-hydroxybutric acid), poly(d-lactic acid), poly(dl-lactic acid), poly(l-lactic acid) (PLLA), poly(lactide/glycolide) copolymers, poly(hydroxyvalerate), poly(hydroxy-varelate-co-hydroxybutyrate), or other PHAs, or other resorbable materials, e.g., protein cell matrices, plant and carbohydrate derivatives (sugars). Resorbable polymers of the present invention can be homopolymers, copolymers or a blend of two or more homopolymers or copolymers. Resorbable polymers of the present invention can have any molecular architecture and can be linear, branched, hyper-branched or dendritic, preferably they are linear or branched. Preferred resorbable polymers include linear resorbable polymers that exhibit cohesive energy less than about 50,000 KJ/Kmol.

The molecular weight of the polymer effects the mechanical properties of the resulting stent. Resorbable polymers for use in the present invention can have any molecular weight. For example, resorbable polymers can range from a single repeat unit to about 10 million repeat units. More specifically, resorbable polymers can have molecular weights of about 10 Daltons to about 100,000,000 Daltons. Preferably, the polymers have intrinsic viscosity (I.V.) greater than about 0.8 dl/g. Intrinsic viscosity can be measured by any method known to one skilled in the relevant art. For example, a viscometer is used in accordance with methods well known in the art. By way of example, a VISCOLAB viscometer can be used, available from Cambridge Applied Systems, Inc. (Medford, Mass., 02155 U.S.A.). Resorbable stents can comprise polymer compositions having a range or specific combination of ranges of molecular weights. Resorbable stents of the present invention comprise a single polymer, or alternatively, a blend of two or more different polymers.

The stent of the present invention optionally comprises a solvent. Any solvent or fluid can be used. The solubility parameter of the solvent is preferably about equal to the solubility parameter of the resorbable polymer. The solubility parameter is a numerical value that indicates the relative solvency behavior of a specific solvent. Solubility parameters for many solvents are well known in the art. The solvent is selected so that it reduces entanglement in the resorbable polymer. For example, a stent comprising poly(l-lactic acid) can be made from a polymer mixture comprising ethyl acetate. The ethyl acetate is used in reducing the entanglement in poly(l-lactic acid) polymer chains.

The solvent optionally comprises a plasticizer. Alternatively, the solvent is a plasticizer. Plasticizer is used herein to mean any material that can decrease the flexural modulus of a polymer. The plasticizer can influence the morphology of the polymer and can affect the melting temperature and glass transition temperature. Examples of plasticizers include, but are not limited to: small organic and inorganic molecules, alcohols, alkyl esters, aliphatic diols, oliogomers of poly(ethylene glycol), phosphate esters of an alcohol, oligomers and small molecular weight polymers (those having molecular weight less than about 50,000), highly-branched polymers and dendrimers. Specific examples include: ethyl acetate, n-propyl acetate, n-butyl acetate, ethylene glycol, diethylene glycol, triethylene glycol, 2-ethylhexanol, isononyl alcohol, isodecyl alcohol, sorbitol, mannitol, oligomeric ethers such as oligomers of polyethylene glycol, including PEG-500, PEG 1000 or PEG-2000 and other biocompatible plasticizers.

The resorbable stent optionally further comprises a modifier. Modifier is used herein to refer to any material added to the polymer to affect the polymer's and stent's properties. Examples of modifiers for use in the invention include resorbable fillers, antioxidants, colorants, crosslinking agents and impact strength modifiers. The drugs and biologically active compounds and molecules.

The resorbable stent optionally further comprises a biologically active agent or drug. The agent or drug will be introduced into the body lumen as the stent is resorbed. Agents or drugs for use in the present invention include but are not limited to antiplatelet agents, calcium agonists, calcium antagonists, anticoagulant agents, antimitotic agents, antioxidants, antimetabolites, antithrombotic agents, anti-inflammatory agents, antiproliferative drugs, hypolipidemic drugs and angiogenic factors. Specific examples include but are not limited to glucocorticoids (e.g. dexamethasone, betamethasone), fibrin, heparin, hirudin, tocopherol, angiopeptin, aspirin, ACE inhibitors, growth factors and oligonucleotides.

Molecular orientation or alignment also effects the degradation rate of the material, and therefore, can effect the elution rate or release of a biological agent or drug. By introducing molecular alignment in the material, the elution rate of a drug will improve, allowing for the more controlled dosing of the patient.

Resorbable stents of the present invention can have any shape, geometry or construction. It is understood by one of ordinary skill in the art that the present invention is not limited to any one type of stent, but that the present invention can be applied to a variety of stent designs. By way of example, the present invention can be applied to the stent designs disclosed in U.S. Pat. No. 6,613,079; U.S. Pat. No. 6,331,189; U.S. Pat. No. 6,287,336; U.S. Pat. No. 6,156,062; U.S. Pat. No. 6,113,621; U.S. Pat. No. 5,984,963; U.S. Pat. No. 5,843,168, which are incorporated herein by reference.

In another embodiment, the present invention relates to a method of manufacturing a minimal injury resorbable stent. The process comprises contacting a resorbable polymer with a solvent to form a polymer mixture, extruding said polymer mixture to form an extrudate, drawing said extrudate to a draw ratio in the range of about 7-350 to form a drawn extrudate and forming said stent from said drawn extrudate.

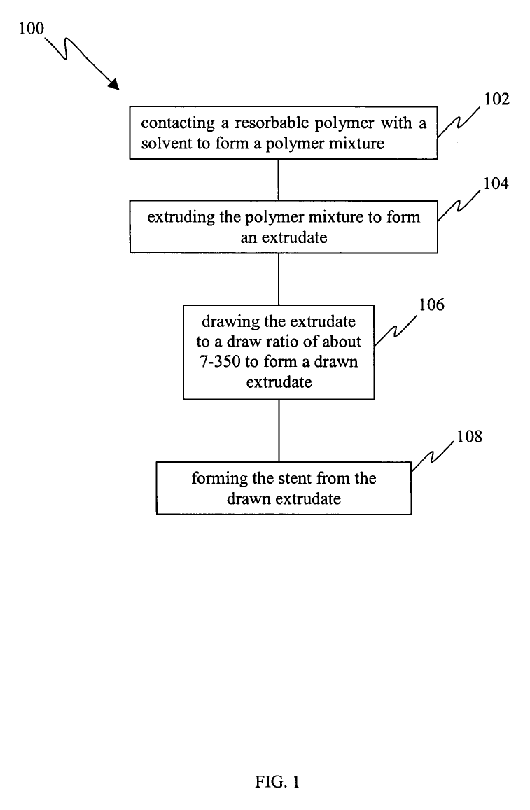

FIG. 1 shows flowchart 100, which depicts steps of the method of the present invention. Flowchart 100 begins with step 102. In step 102, a resorbable polymer is contacted with a solvent to form a polymer mixture. The solvent can comprise a plasticizer, or alternatively, is a plasticizer. The solvent is chosen such that it reduces the entanglement of polymer chains. Any solvent can be used. Preferably, the solvent or plasticizer has a solubility parameter about equal to the solubility parameter of the resorbable polymer. Any polymer concentration can be used, so long as the polymer concentration results in reduction of the entanglement density of the polymer chains during extrusion. Preferably, the polymer concentration is about 10-99 wt %. The polymer mixture can optionally further comprise additives and modifiers, including biologically active agents and drugs.

Referring back to FIG. 1, step 104 follows step 102. In step 104, the polymer mixture is extruded to form an extrudate. The process of extruding a mixture to form an extrudate is well known to one skilled in the relevant art. Any method of extrusion can be used to provide an extrudate. The extrudate can be any geometry, shape or size, specific examples include, but are not limited to sheets and tubes.

An extrudate in the form of a sheet can be produced by any extrusion method known to one skilled in the relevant art. For example, the polymer mixture can be extruded through a flat die over a casting roll, through an annular die onto a sizing mandrel, between two or more rolls in a calendering process or by some other extrusion process. The temperature of the die and roll can be independently varied and controlled, preferably, the temperature of the extrusion is such that a reduction in the chain entanglement of the resorbable polymer results. For example, a polymer mixture comprising poly(l-lactic acid) mixtures can be extruded through a die or calendered between rolls at a temperature about 75-250° C. In another example, a polymer mixture comprising poly(glycolic acid) mixtures can be extruded through a die or calendered between rolls at a temperature about 75-250° C. The extruded sheet can be cooled in a bath with a suitable fluid such as water or in air. This process provides an extrudate in the form of a sheet. The particular extrusion method and parameters used during the extrusion process would be apparent to one skilled in the relevant art.

An extrudate in the form of a tube can be produced by any extrusion method known to one skilled in the relevant art. Examples of extruders for use in the invention include single screw and double screw extruders that produce tube-shaped extrudates. The extrusion temperature is controlled such that a reduction in the chain entanglement of the resorbable polymer results. For example, a polymer mixture comprising poly(l-lactic acid) is extruded at a temperature about 75-250° C. In another example, a polymer mixture comprising poly(glycolic acid) is extruded at a temperature about 75-250° C. The tubular extrudate can be cooled in a bath with a suitable fluid such as water or in air. The tubular extrudate is a hollow cylindrical-shaped tube having a longitudinal axis.

Referring back to FIG. 1, step 106 follows step 104. In step 106, the extrudate is drawn to a draw ratio of about 7-350 to form a drawn extrudate. Alternatively, the extrudate is drawn to a draw ratio of about 10-300. The drawing step, 106, introduces molecular alignment or orientation into the extrudate. Any method of drawing can be used for drawing the extrudate.

One particular example, for drawing an extrudate in the form of a sheet, involves stretching the extruded sheet at a controlled temperature and controlled rate. The temperature and rate can be any temperature and rate that result in the introduction of molecular alignment in the extruded sheet. Preferably, the temperature is between the glass transition temperature and the melting temperature of the polymer mixture comprising the resorbable polymer. For example, an extruded sheet comprising poly(l-lactic acid) is stretched at a temperature about 75-250° C. Any method can be used to stretch the sheet. For example, a machine is used, such as the Lab Stretcher Karo IV®, available from Brückner, in Schweinbach, Germany. The stretching process can be performed uniaxially or biaxially. Uniaxial stretching produces substantially uniaxial molecular orientation, whereas biaxial stretching produces biaxial molecular orientation. Biaxial stretching is performed sequentially, or alternatively, simultaneously. Bulk sheet properties such as sheet thickness are also controlled during the stretching process. Preferably, the sheet is stretched uniaxially to induce the maximum increase in tensile strength and modulus in the stretch direction. The draw ratio measures the relative degree of stretching between the stretched sheet and unstretched sheet. In the present invention, draw ratios can range from about 7 to about 350, alternatively about 10-300. The higher the draw ratio, the greater the amount of molecular alignment, and therefore, the greater the increase in tensile strength and modulus of the resorbable material. The amount of molecular alignment can be monitored before, during and after the stretching. Any method of monitoring the level of orientation can be used. For example, FTIR is used, as is well known to one skilled in the relevant art. This process provides a drawn extrudate in the form of a sheet.

Molecular alignment can also be introduced in a tubular extrudate during drawing step 106. Any method known to one of ordinary skill in the art can be used to draw or introduce molecular alignment in the tubular extrudate. One particular example involves introducing radial molecular alignment by blow-molding the tube at a temperature approximately between the glass transition temperature and the melting temperature of the polymer mixture comprising the resorbable polymer. For example, a tubular extrudate comprising poly(l-lactic acid) is radially expanded or blow-molded at a temperature about 75-250° C. Any method of blow-molding the tubular extrudate can be used to induce the molecular alignment. In one example, a tubular extrudate is placed in a blow-molding machine and radially expanded. A suitable medium is used to expand the extrudate. Suitable medium can be a gas or liquid, or there can be no medium and the expansion is performed mechanically. The molecular alignment in the extrudate is related to the amount of expansion or draw ratio. In the present invention, draw ratios can range from about 7 to about 350, alternatively about 10-300. The greater the amount of expansion, the greater the amount of orientation and the greater the increase in tensile strength and modulus. Any method of monitoring the level of orientation can be used. For example, FTIR is used, as is well known to one skilled in the relevant art.

Referring back to FIG. 1, step 108 follows step 106. In step 108, a stent is formed from the drawn extrudate. Any method known to one of ordinary skill in the art can be used to produce the stent. For example, the drawn extrudate may be used to design a stent comprising a ratcheting mechanism. Any type of ratcheting mechanism may be used. One example of a ratcheting mechanism for use in the present invention is disclosed in U.S. Pat. No. 5,984,963. In another example, the drawn extrudate may be used to design a stent in the form of a spiral. One example of a spiral-formed stent for use in the present invention is disclosed in U.S. Pat. No. 6,156,062. A ratcheting mechanism can be introduced into drawn extrudate using a laser machining process, by using a pre-shaped die, or any other method known to one of ordinary skill in the art. A locking mechanism can also be introduced into the stent using the same methods listed above for introducing the ratcheting mechanism. The ratcheting and locking mechanisms help to further enhance the recoil resistance of the low profile resorbable stents. In a further example, a low profile resorbable stent is formed from a tubular drawn extrudate. The stent can be formed using any method known to one of ordinary skill in the art, for example, the tube is laser machined to a desired geometry, such as those listed above.

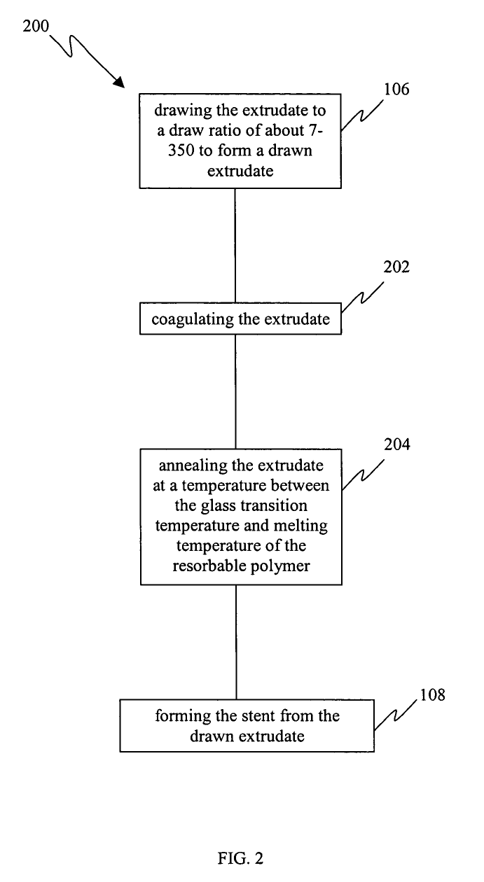

In another embodiment, additional, optional steps are performed in the method of the present invention. As shown in flowchart 200 of FIG. 2, step 202 can be performed after drawing the extrudate in step 106. In step 202, the drawn extrudate is coagulated. Any method known to one skilled in the relevant art can be used to coagulate the drawn extrudate. For example, a vacuum oven is used to remove the solvent. Alternatively, the drawn extrudate is coagulated in a suitable fluid. Suitable fluids include those fluids that remove the solvent from the extrudate without dissolving the extrudate, for example a C₁-C₆ alcohol.

Referring back to FIG. 2, step 204 optionally follows step 202. In step 204, the drawn and coagulated extrudate is annealed at a temperature approximately between the glass transition temperature and melting temperature of the resorbable polymer. Any method known to one of skill in the relevant art can be used to anneal the extrudate. For example, the extrudate is annealed in an oven. The extrudate can optionally be annealed in an oven under controlled conditions. For example, the extrudate is annealed in an oven under nitrogen, under a vacuum, or under positive pressure above atmospheric pressure. Annealing the extrudate may serve to enhance the strength of the resorbable material even further and help to stabilize the properties. After the extrudate is annealed in step 204, the extrudate can be used to form a stent, as described above, in step 108.

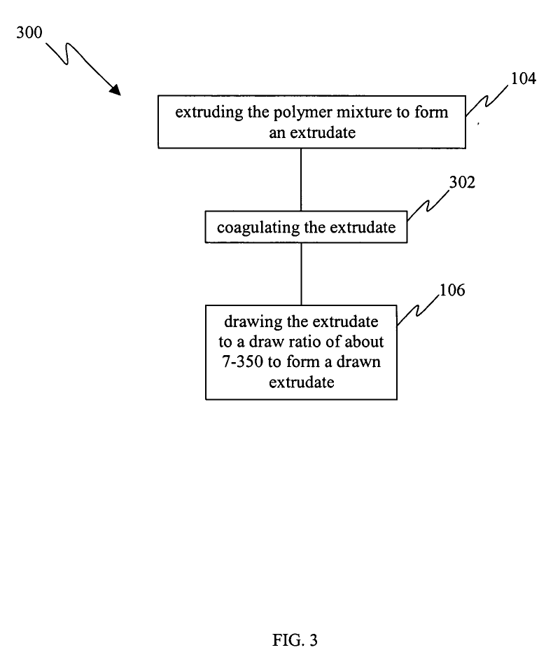

In another embodiment, other optional steps can be performed in the method of the present invention. As shown in flowchart 300 in FIG. 3, step 302 can be performed after extruding the polymer mixture in step 104. In optional step 302, the extrudate is coagulated. The methods used to effect step 302 can be can be the same, or optionally, they can be similar or different from the methods previously described in step 202.

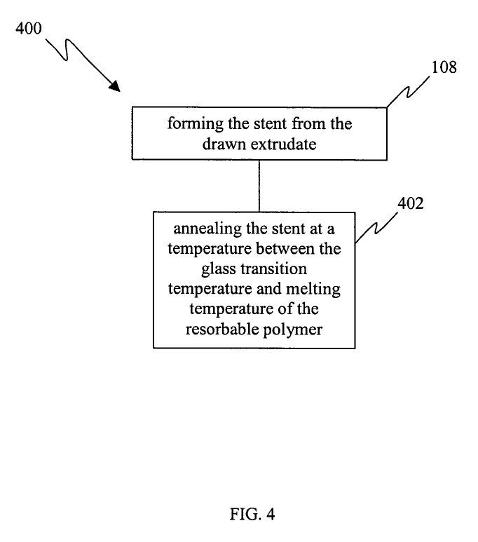

In another embodiment, FIG. 4 shows flowchart 400 with optional step 402. Step 402 optionally follows step 108. In step 402, the stent is annealed at temperature approximately between the glass transition temperature and melting temperature of the resorbable polymer. Annealing step 402 is similar to step 204, described above, in that similar methods can be used to effect the annealing. The annealing steps 204 and 402 can be the same, or optionally, they can be similar or different.

It is understood to one skilled in the relevant art that any combination of optional and additional steps can be used in practicing the methods of the present invention. For example, no optional steps are performed. Alternatively, all steps, as shown in FIGS. 1-4, are performed.

The methods of the present invention can be used to manufacture stents having enhanced properties. The methods of the present invention provide resorbable polymeric materials having significantly enhanced tensile strengths and modulus. These materials can be used to manufacture resorbable stents having low profile that will induce minimal injury when deployed in the patient. For example, resorbable stents made from poly(l-lactic acid) have in the past been made from poly(l-lactic acid) polymers with tensile strengths of only 50-60 MPa. In accordance with the present invention, high molecular weight poly(l-lactic acid) (mean M_{w}>70,000 g/mol) mixtures comprising ethyl acetate can be drawn to ratios about 7-350 and can have tensile strengths greater than 300 MPa. This increase in tensile strength can lead to equivalent reduction in stent strut area in contact with the vessel wall and can therefore serve to reduce the level of vessel injury. The high level of molecular alignment can also serve to retard the degradation process. This is important because minimal injury resorbable stents comprising a biologically active drug can have more controlled and improved dosing profiles as the stent will resorb at a more controlled rate.

It will be understood by those skilled in the art that various changes in form and details may be made therein without departing from the spirit and scope of the present invention as defined in the appended claims. Thus, the breadth and scope of the present invention should not be limited by any of the above-described exemplary embodiments, but should be defined only in accordance with the following claims and their equivalents.

1. A minimal injury resorbable stent comprising an oriented resorbable material drawn to a ratio about 7-350.

2. The stent of claim 1, wherein said material is drawn to a ratio of about 10-350.

3. The stent of claim 1, wherein said material is gel-extruded.

4. The stent of claim 1, wherein said material has tensile strength about 50-500 MPa.

5. The stent of claim 4, wherein said material has tensile strength about 75-400 MPa.

6. The stent of claim 5, wherein said material has tensile strength about 100-300 MPa.

7. The stent of claim 1, wherein said material has Young's Modulus about 2-300 GPa and tensile strength about 50-500 MPa.

8. The stent of claim 1, wherein said material is a polyester, polyanhydride, polyamide, polyurethane, polyurea, polyether, polysaccharide, polyamine, polyphosphate, polyphosphonate, polysulfonate, polysulfonamide, polyphosphazene, hydrogel, polylactide, polyglycolide, protein cell matrix, or copolymer or polymer blend therof.

9. The stent of claim 10, wherein said material is fibrin, collagen, polycaprolactone, poly(glycolic acid), poly(l-lactic acid), poly(3-hydroxybutric acid), poly(dl-lactic acid), poly(d-lactic acid), poly(lactide/glycolide) copolymers, poly(hydroxyvalerate) or poly(hydroxyvarelate-co-hydroxybutyrate).

10. The stent of claim 1, further comprising a biologically active agent.

11. The stent of claim 13, wherein said agent is an antiplatelet agent, calcium agonist, calcium antagonist, anticoagulant agent, antimitotic agent, antioxidant, antimetabolite, antithrombotic agent, anti-inflammatory agent, antiproliferative drug, hypolipidemic drug, angiogenic factor, glucocorticoid, dexamethasone, betamethasone, fibrin, heparin, hirudin, tocopherol, angiopeptin, aspirin, ACE inhibitor, growth factors or oligonucleotide.

12. The stent of claim 1, further comprising a solvent.

13. The stent of claim 12, wherein said material is poly(l-lactic acid) and said solvent is ethyl acetate.

14. A method of manufacturing a minimal injury resorbable stent, the process comprising:
(a) contacting a resorbable polymer with a solvent to form a polymer mixture;
(b) extruding said polymer mixture to form an extrudate;
(c) drawing said extrudate to a draw ratio of about 7-350 to form a drawn extrudate; and
(d) forming said stent from said drawn extrudate.

15. The method of claim 14, wherein said drawn extrudate has tensile strength about 50-500 MPa.

16. The method of claim 14, wherein said drawn extrudate has tensile strength about 75-400 MPa.

17. The method of claim 14, wherein said drawn extrudate has tensile strength about 100-350 MPa.

18. The method of claim 14, wherein said resorbable polymer is fibrin, collagen, polycaprolactone, poly(glycolic acid), poly(l-lactic acid), poly(3-hydroxybutric acid), poly(dl-lactic acid), poly(d-lactic acid), poly(lactide/glycolide) copolymers, poly(hydroxyvalerate) or poly(hydroxyvarelate-co-hydroxybutyrate).

19. The method of claim 14, wherein said drawing further comprises:
drawing said extrudate at a temperature between the glass transition temperature and melting temperature of said resorbable polymer.

20. The method of claim 14, further comprising:
coagulating said extrudate.

21. The method of claim 14, further comprising:
annealing said extrudate at a temperature between the glass transition temperature and melting temperature of said resorbable polymer.
